(19) United States
(12) Patent Application Publication
(10) Pub. No.: US 2025/0032329 A1
(43) Pub. Date: Jan. 30, 2025

(54) POINT BONDED EXTRUSION LAMINATES

(71) Applicant: Berry Global, Inc., Evansville, IN (US)

(72) Inventors: Iyad Muslet, West Chester, OH (US); Nathan Howell Hackler, Loveland, OH (US)

(21) Appl. No.: 18/785,728

(22) Filed: Jul. 26, 2024

Related U.S. Application Data
(60) Provisional application No. 63/529,178, filed on Jul. 27, 2023.

(51) Int. Cl. A61F 13/49; A61F 13/15; B32B 5/02; B32B 7/14; B32B 27/08; B32B 27/12

(52) U.S. Cl. CPC A61F 13/49015; A61F 13/15699; A61F 13/49012; B32B 5/022; B32B 7/14; B32B 27/08; B32B 27/12; A61F 2013/49033; B32B 2307/536; B32B 2307/54; B32B 2307/718; B32B 2307/724; B32B 2307/726; B32B 2307/7376; B32B 2555/02

(57) ABSTRACT

Point bonded extrusion laminates (PEL) are provided. The PELs include at least a first nonwoven layer and an elastic film that have been laminated together via a first plurality of discrete bond sites bonding the first nonwoven layer to a first side of the elastic film. The first plurality of discrete bond sites define a first bonded area and a first bond pattern between the first nonwoven layer and the first side of the elastic film. The PELs also include first nonbonded portions of the first nonwoven layer located between the first plurality of discrete bond sites, in which the first nonbonded portions of the first nonwoven layer are not bonded to the first side of the elastic film.

### TECHNICAL FIELD

Embodiments of the presently-disclosed invention relate generally to point bonded extrusion laminates (PEL) including at least a first nonwoven layer and an elastic film that have been laminated together via a first plurality of discrete bond sites. The PEL also includes first nonbonded portions of the first nonwoven layer that extend out of an x-y plane of the PEL to impart a desired rugose outer surface to the PEL.

### BACKGROUND

Disposable absorbent articles, such as diapers, are used to collect and store bodily exudates such as urine and feces from wearers, such as babies. To prevent undesired leakage, absorbent articles should fit well against the wearer and be easily secured. For example, it may be desirable for certain portions of a disposable absorbent article to provide a level of elasticity to provide comfort to a variety of wearers, and/or for the ease of fastening.

### SUMMARY OF INVENTION

One or more embodiments of the invention may address one or more of the aforementioned problems. Certain embodiments according to the invention provide a point bonded extrusion laminate (PEL) including at least a first nonwoven layer and an elastic film that have been laminated together via a first plurality of discrete bond sites bonding the first nonwoven layer to a first side of the elastic film. The first plurality of discrete bond sites define a first bonded area and a first bond pattern between the first nonwoven layer and the first side of the elastic film. The PELs also include first nonbonded portions of the first nonwoven layer located between the first plurality of discrete bond sites, in which the first nonbonded portions of the first nonwoven layer are not bonded to the first side of the elastic film.

In another aspect, the present invention provides a disposable absorbent article including at least one component thereof comprising a PEL, such as described and disclosed herein. The at least one component thereof may comprise an elastic ear, a panel, or a waistband, for example, of a diaper.

In another aspect, the present invention provides a method of making a PEL, such as those described and disclosed herein. The method may comprise the following steps: (a) providing or forming a first nonwoven layer; (b) melt extruding an elastic film onto the first nonwoven layer; (c) laminating the elastic film onto the first nonwoven layer by passing the elastic film and the first nonwoven layer through a roller nip comprising a point bond nip roll to form an intermediate material, wherein the point bond nip roll includes a first plurality of discrete protrusions that form a first plurality of discrete bond sites between the first nonwoven layer and a first side of the elastic film, and wherein the first plurality of discrete bond sites define a first bonded area and a first bond pattern between the first nonwoven layer and the first side of the elastic film, and wherein first nonbonded portions of the first nonwoven layer located between the first plurality of discrete bond sites are not bonded to the first side of the elastic film; and (d) activating the intermediate material to form the PEL, wherein activating the intermediate material forms deformed portions that project outwardly from an x-y plane.

### BRIEF DESCRIPTION OF THE DRAWING(S)

The invention now will be described more fully hereinafter with reference to the accompanying drawings, in which some, but not all embodiments of the invention are shown. Indeed, this invention may be embodied in many different forms and should not be construed as limited to the embodiments set forth herein; rather, these embodiments are provided so that this disclosure will satisfy applicable legal requirements. Like numbers refer to like elements throughout, and wherein:

FIG. 1 illustrates a PEL including a first nonwoven layer and an elastic film in accordance with certain embodiments of the invention;

FIG. 2 illustrates a PEL including an elastic film disposed between a first nonwoven layer and a second nonwoven layer in accordance with certain embodiments of the invention;

FIG. 3A illustrates a prior art extrusion laminate having a continuous or total bond (e.g., area bonded as opposed to point bonded) formed from a smooth surface or flat surface roll, in which the extrusion laminate is devoid of nonbonded regions.

FIG. 3B illustrates a PEL in accordance with certain embodiments of the invention in which the nonbonded areas of the nonwoven layer have formed deformed portions or regions extending out of the z-y plane of the PEL to provide rogusity (e.g., variations of amplitude in height of the surface defined by the nonwoven layer), in accordance with certain embodiments of the invention;

FIG. 4 illustrates a point bond nip roll having a plurality of protrusions that may be used to form the PEL, in accordance with certain embodiments of the invention;

FIG. 5 is a schematic illustrating the impact of an activation step to form a PEL from an intermediate material, in accordance with certain embodiments of the invention;

FIG. 6 illustrates a load vs. stain graph for a PEL and comparative laminates that are commercially used as elastic ears or panels of disposable absorbent article (e.g., diaper);

FIG. 7 illustrates a load vs. stain graph for a PEL and comparative laminates that are commercially used as waistbands of disposable absorbent article (e.g., diaper);

FIG. 8 illustrates a tensile strength vs. strain graph for a PEL and comparative laminates that are commercially used as waistbands of disposable absorbent article (e.g., diaper);

FIG. 9 illustrates a load vs. stain graph for a PEL and comparative laminates that are commercially used as waistbands of disposable absorbent article (e.g., diaper); and

FIG. 10 illustrates a tensile strength vs. strain graph for a PEL and comparative laminates that are commercially used as waistbands of disposable absorbent article (e.g., diaper).

### DETAILED DESCRIPTION

The invention now will be described more fully hereinafter with reference to the accompanying drawings, in which some, but not all embodiments of the invention are shown. Indeed, this invention may be embodied in many different forms and should not be construed as limited to the embodiments set forth herein; rather, these embodiments are provided so that this disclosure will satisfy applicable legal requirements. As used in the specification, and in the appended claims, the singular forms “a”, “an”, “the”, include plural referents unless the context clearly dictates otherwise.

The presently-disclosed invention relates generally to PELs and methods of making PELs that may be suitable for use in disposable absorbent articles, such as baby and/or adult diapers. For example, the PELs may be used alone or in conjunction with other materials to form elastic waistbands, clastic panels, and/or clastic cars. The PELs utilize a plurality of discrete (e.g., separate and individual) bond sites between one or more nonwoven layers and an elastic film. Such embodiments, for instance, may increase stretchability, softness, and/or rousity of the outer surface(s) of the PEL. In this regard, the one or more nonwoven layers include a plurality of nonbonded portions where the fibers of the one or more nonwoven layers have at least partially been deformed, such as by an activation step, to provide a rugose outer surface to the PEL having a plurality of deformed portions that extend outwardly from an x-y plane of the PEL. In accordance with certain embodiments of the invention, the formation of the PEL may beneficially combine film extrusion and lamination in a single step. In accordance with certain embodiments of the invention, the PEL may include a plurality of apertures extending through the PEL or be devoid of apertures extending through the PEL. Additionally or alternatively, the PEL may be devoid of an adhesive, such as between the elastic film and a nonwoven layer.

The terms “substantial” or “substantially” may encompass the whole amount as specified, according to certain embodiments of the invention, or largely but not the whole amount specified (e.g., 95%, 96%, 97%, 98%, or 99% of the whole amount specified) according to other embodiments of the invention.

The terms “polymer” or “polymeric”, as used interchangeably herein, may comprise homopolymers, copolymers, such as, for example, block, graft, random, and alternating copolymers, terpolymers, etc., and blends and modifications thereof. Furthermore, unless otherwise specifically limited, the term “polymer” or “polymeric” shall include all possible structural isomers; stereoisomers including, without limitation, geometric isomers, optical isomers or enantionmers; and/or any chiral molecular configuration of such polymer or polymeric material. These configurations include, but are not limited to, isotactic, syndiotactic, and atactic configurations of such polymer or polymeric material. The term “polymer” or “polymeric” shall also include polymers made from various catalyst systems including, without limitation, the Ziegler-Natta catalyst system and the metallocene/single-site catalyst system. The term “polymer” or “polymeric” shall also include, in according to certain embodiments of the invention, polymers produced by fermentation process or biosourced.

The terms “nonwoven” and “nonwoven web”, as used herein, may comprise a web having a structure of individual fibers, fibers, and/or threads that are interlaid but not in an identifiable repeating manner as in a knitted or woven fabric. Nonwoven fabrics or webs, according to certain embodiments of the invention, may be formed by any process conventionally known in the art such as, for example, meltblowing processes, spunbonding processes, needle-punching, hydroentangling, air-laid, and bonded carded web processes. A “nonwoven web”, as used herein, may comprise a plurality of individual fibers that have not been subjected to a consolidating process. In certain instances, the “nonwoven web” may comprises a plurality of layers, such as one or more spunbond layers and/or one or more meltblown layers. For instance, a “nonwoven web” may comprises a spunbond-meltblown-spunbond structure.

The terms “fabric” and “nonwoven fabric”, as used herein, may comprise a web of fibers in which a plurality of the fibers are mechanically entangled or interconnected, fused together, and/or chemically bonded together. For example, a nonwoven web of individually laid fibers may be subjected to a bonding or consolidation process to bond at least a portion of the individually fibers together to form a coherent (e.g., united) web of interconnected fibers.

The term “layer”, as used herein, may comprise a generally recognizable combination of similar material types and/or functions existing in the X-Y plane.

The term “consolidated” and “consolidation”, as used herein, may comprise the bringing together of at least a portion of the fibers of a nonwoven web into closer proximity or attachment there-between (e.g., thermally fused together, chemically bonded together, and/or mechanically entangled together) to form a bonding site, or bonding sites, which function to increase the resistance to external forces (e.g., abrasion and tensile forces), as compared to the unconsolidated web. The bonding site or bonding sites, for example, may comprise a discrete or localized region of the web material that has been softened or melted and optionally subsequently or simultaneously compressed to form a discrete or localized deformation in the web material. Furthermore, the term “consolidated” may comprise an entire nonwoven web that has been processed such that at least a portion of the fibers are brought into closer proximity or attachment there-between (e.g., thermally fused together, chemically bonded together, and/or mechanically entangled together), such as by thermal bonding or mechanical entanglement (e.g., hydroentanglement) as merely a few examples. Furthermore, the term “consolidated” and “consolidation” may comprise the bonding by means of a through-air-bonding operation. The term “through-air bonded” and “though-air-bonding”, as used herein, may comprise a nonwoven web consolidated by a bonding process in which hot air is used to fuse the fibers at the surface of the web and optionally internally within the web. By way of example only, hot air can either be blown through the web in a conveyorized oven or sucked through the web as it passes over a porous drum as a vacuum is developed. The temperature of and the rate of hot air are parameters that may determine the level or the extent of bonding in nonwoven web. In accordance with certain embodiments of the invention, the temperature of the hot air may be high enough to melt, induce flowing, and/or fuse the a plurality of fibers having a lower melting point temperature or onset of lower melting point temperature (e.g., amorphous fibers) to a plurality of fibers having a higher melting point temperature or onset of lower melting point temperature (e.g., semi-crystalline or crystalline fibers). Such a web may be considered a “consolidated nonwoven”, “nonwoven fabric” or simply as a “fabric” according to certain embodiments of the invention.

As used herein, “meltspun” or “melt-spun” generally refers to fiber forming processes of spunbonding or melt-blowing.

The term “spunbond”, as used herein, may comprise fibers which are formed by extruding molten thermoplastic material as fibers from a plurality of fine, usually circular, capillaries of a spinneret with the diameter of the extruded fibers then being rapidly reduced. According to an embodiment of the invention, spunbond fibers are generally not tacky when they are deposited onto a collecting surface and may be generally continuous as disclosed and described herein. It is noted that the spunbond used in certain composites of the invention may include a nonwoven described in the literature as SPINLACE®. Spunbond fibers, for example, comprise continuous fibers.

As used herein, the term “continuous fibers” refers to fibers which are not cut from their original length prior to being formed into a nonwoven web or nonwoven fabric. Continuous fibers may have average lengths ranging from greater than about 15 centimeters to more than one meter, and up to the length of the web or fabric being formed. For example, a continuous fiber, as used herein, may comprise a fiber in which the length of the fiber is at least 1,000 times larger than the average diameter of the fiber, such as the length of the fiber being at least about 5,000, 10,000, 50,000, or 100,000 times larger than the average diameter of the fiber.

The term “activated” or “activating”, as used herein, may comprise a material that has been mechanically deformed or process that mechanically deforms, for example by incremental stretching, in order to increase the extensibility of at least a portion of the material. In one embodiment, for example, a material (e.g., nonwoven web or fabric) may be activated by, for example, incrementally stretching the material in at least one direction.

The term “incremental stretching”, as used herein, may comprise a process in which a web or fabric is supported at closely spaced apart locations and then the unsupported segments of the web or fabric between these closely spaced apart locations are stretched. A non-limiting example of incremental stretching rolls designed for machine direction and cross direction stretching can be found in U.S. Pat. No. 4,223,059, the contents of which are hereby incorporated herein by reference. Incremental stretching, according to one embodiment, may comprise a process performed by embossing of the web as illustrated in U.S. Patent Application No. 2014/0276517, the contents of which are hereby incorporated herein by reference.

The term “machine direction” or “MD”, as used herein, comprises the direction in which the fabric produced or conveyed. The term “cross-direction” or “CD”, as used herein, comprises the direction of the fabric substantially perpendicular to the MD.

The term “elastic”, “elastomer” or “elastomeric”, as used interchangeably herein, may comprise a material that when stretched and released will recover to near its original length (e.g., return to within 20%, 10%, 5%, 3%, or 1% of its original length). The term “elastic”, “elastomer”, or “elastomeric”, as used interchangeably herein, may also comprise a material that exhibits the ability to be stretched and released several times and, to exert repetitively the same or just slightly lower force when stretched at the same extension level. Elastic materials, for example, may comprise elastomers, such as elastomeric polymers. Non-limiting exemplary elastomers may comprise, according to certain embodiments, one or more elastomers such as an acrylate; a polyolefin, such as polyethylene, polypropylene, polybutylene, polyhexene, polyoctene; polystyrenes; polyurethanes; polyesters, such as polyethyleneterephthalate; polyamides such as nylon; natural or synthetic rubber resins such as styrenic block copolymers (e.g., styrene-isoprene-styrene, styrene-butadiene-styrene, styrene-ethylene-ethylene-propylene-styrene copolymers, styrene-ethylene-butylene-styrene); epoxies; vinyl acetates, such as ethylene vinyl acetate; polydiorganosiloxane polyurea copolymers; copolymers thereof and mixtures thereof. Additionally or alternatively, non-limiting elastomers may include elastomeric polyolefins (e.g., VISTAMAXX™ from ExxonMobil Chemical Company, VERSIFY™, a propylene-ethylene elastomeric polymer, and AFFINITY™ from The Dow Chemical Company), polyether block amide copolymer (e.g., PEBAX® from Arkema Group), polyester block amide copolymer, copolyester thermoplastic elastomer (e.g., ARNITEL® from DSM Engineering Plastics, HYTREL® from E.I. DuPont de Nemours and Company), thermoplastic urethane elastomer, and/or combinations thereof. In certain embodiments, example elastomers may comprise VISTAMAXX™ propylene-based elastomers (commercially available form ExxonMobile), which comprise copolymers of propylene and ethylene. VISTAMAXX™ propylene-based elastomers, for example, comprise isotactic polypropylene microcrystalline regions and random amorphous regions.

Certain embodiments according to the invention provide a point bonded extrusion laminate (PEL) including at least a first nonwoven layer and an elastic film that have been laminated together via a first plurality of discrete bond sites bonding the first nonwoven layer to a first side of the elastic film. The first plurality of discrete bond sites define a first bonded area and a first bond pattern between the first nonwoven layer and the first side of the elastic film. The PELs also include first nonbonded portions of the first nonwoven layer located between the first plurality of discrete bond sites, in which the first nonbonded portions of the first nonwoven layer are not bonded to the first side of the elastic film. In accordance with certain embodiments of the invention, the PEL further comprises a second nonwoven layer. For example, the elastic film is located between and adjacent the first nonwoven layer and the second nonwoven layer. The PEL may also comprise a second plurality of discrete bond sites bonding the second nonwoven layer to a second side of the elastic film. The plurality of second discrete bond sites may define a second bonded area and a second bond pattern between the second nonwoven layer and the second side of the elastic film. The PEL may also include second nonbonded portions of the second nonwoven layer located between the second plurality of discrete bond sites, in which the second nonbonded portions of the second nonwoven layer are not bonded to the second side of the clastic film.

In accordance with certain embodiments of the invention, the first nonwoven layer, the second nonwoven layer, or both may comprise a variety of nonwoven webs or nonwoven fabrics, such as a spunbond nonwoven, a carded nonwoven, a meltblown nonwoven, a hydroentangled nonwoven, or any combination thereof. Additionally or alternatively, the first nonwoven layer, the second nonwoven layer, or both comprise a basis weight from about 3 to about 50 gsm, such as at least about any of the following: 3, 5, 9, 10, 12, and 15 gsm, and/or at most about 50, 45, 40, 35, 30, 25, 20, and 15 gam.

In accordance with certain embodiments of the invention, the first nonwoven layer, the second nonwoven layer, or both may comprise a polymeric composition comprising a polymer component and an optional additive component. The polymer component, for example, may comprise one or more polyolefins, such as a polypropylene or copolymer thereof, or polyethylene or copolymer thereof. Additionally or alternatively, the polymer component may comprise one or more polymers produced by a fermentation process or biosourced. For example, the polymer component may comprise one or more polylactic acids (PLA), polyhydroxyalkanoates (PHA), poly (hydroxycarboxylic) acids, or any combinations thereof.

In accordance with certain embodiments of the invention, at least a portion of the first nonbonded portions, the second nonbonded portions, or both comprise deformed portions that project outwardly from an x-y plane. In this regard, the deformed portions may form loops or hills that extend outwardly from the x-y plane of the PEL to provide one or more outer surfaces having visually discernable rugose structure. For example, the deformed portions may have an average height of about 1 to about 10 mm, such as at least about any of the following: 1, 2, 3, 4, and 5 mm, and/or at most about any of the following: 10, 9, 8, 7, 6, and 5 mm.

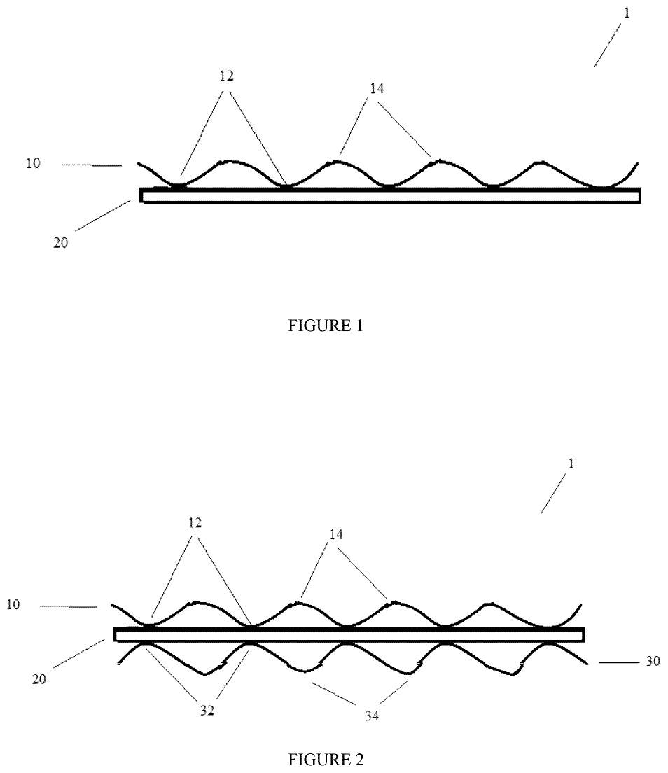

FIG. 1, for instance, illustrates a PEL 1 including a first nonwoven layer 10 and an elastic film 20 in accordance with certain embodiments of the invention. As shown in FIG. 1, a first plurality of bond sites 12 bonding the first nonwoven layer 10 to the elastic film 20 in which at least a portion of first nonbonded portions comprise deformed portions 14 that project outwardly from an x-y plane of the PEL. FIG. 2 illustrates a PEL 1 including an elastic film 20 disposed between a first nonwoven layer 10 and a second nonwoven layer 30. As shown in FIG. 2, a first plurality of bond sites 12 bonding the first nonwoven layer 10 to the elastic film 20 in which at least a portion of first nonbonded portions comprise deformed portions 14 that project outwardly from an x-y plane of the PEL, and a second plurality of bond sites 32 bonding the second nonwoven layer 30 to the elastic film 20 in which at least a portion of second nonbonded portions comprise deformed portions 34 that project outwardly from an x-y plane of the PEL.

As noted above, the PEL moves away from the traditional use of an extrusion laminate having a continuous or total bonded area (e.g., area bonded) between a nonwoven layer and an elastic film. In this regard, for example, the first bonded area, the second bonded area, or both comprise from about 5 to about 40%, such as at least about any of the following: 5, 6, 8, 10, 12, 15, 18, and 20%, and/or at most about any of the following: 40, 38, 35, 32, 30, 28, 25, 22, and 20%. Additionally or alternatively, the first plurality of discrete bond sites, the second plurality of discrete bond sites, or both may have an average bonded area of about from about 0.25 mm² to about 3 mm², such as at most about any of the following: 3, 2.5, 2.25, 2, 1.75, 1.5, 1.25, 1, and 0.75 mm² and/or at least about any of the following: 0.25, 0.3, 0.4, 0.5, 0.6, 0.7, 0.75, 0.8, 0.9, 1, and 1.25 mm².

Additionally or alternatively, the first plurality of discrete bond sites, the second plurality of discrete bond sites, or both may have a round shape, a polygonal shape, a diamond shape, an elliptical shape, or any combination thereof. For instance, the first plurality of discrete bond sites, the second plurality of discrete bond sites, or both may have comprise a combination of different bond sizes and/or different shapes. Additionally or alternatively, the first bond pattern, the second bond patter, or both comprise, for example, a wave pattern, an unaligned diamond pattern, straight line pattern, or any combination thereof.

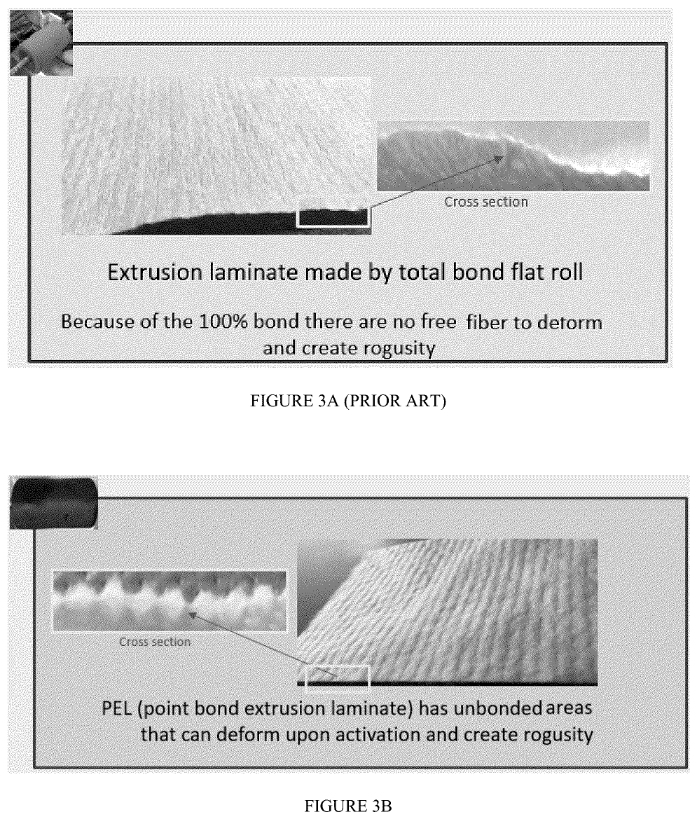

FIG. 3A illustrates a prior art extrusion laminate having a continuous or total bond (e.g., area bonded as opposed to point bonded) formed from a smooth surface or flat surface roll, in which the extrusion laminate is devoid of nonbonded regions. In such extrusion laminates, the nonwoven layer does not include any nonbonded regions to provide free fiber portions for deformation and formation of a rugose surface. To the contrary, FIG. 3B illustrates a PEL in accordance with certain embodiments of the invention in which the nonbonded areas of the nonwoven layer have formed deformed portions or regions extending out of the z-y plane of the PEL to provide rugosity (e.g., variations of amplitude in height of the surface defined by the nonwoven layer), in accordance with certain embodiments of the invention.

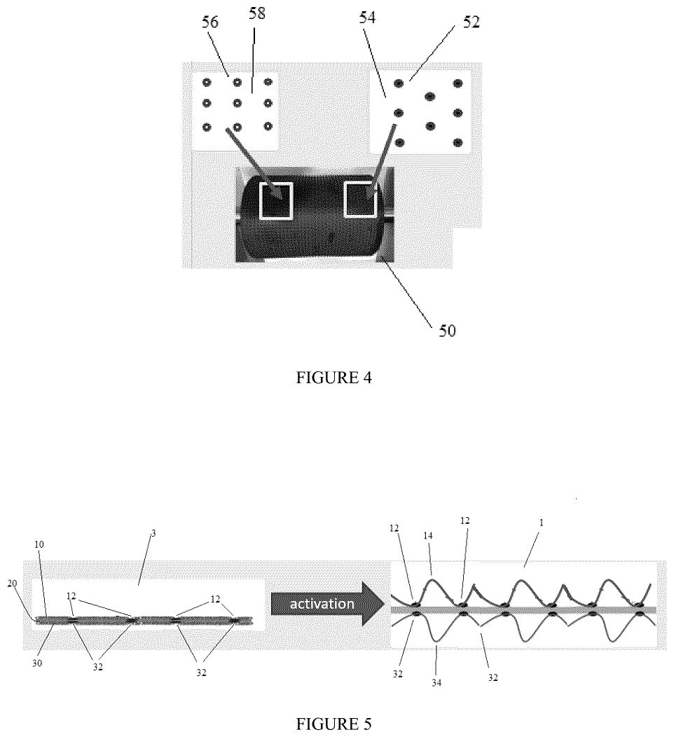

FIG. 4 illustrates a point bond nip roll 50 having a plurality of protrusions 52, 56 extending from a contiguous non-elevated portion 54, 58 that may be used to form the PEL, in accordance with certain embodiments of the invention. The example protrusions 52, 56 illustrate just a couple example configurations or shapes for protrusions.

In accordance with certain embodiments of the invention, the elastic film may comprise an average thickness from about 10 to about 300 microns, such as at least about any of the following: 10, 15, 20, 50, 80, 100, 120, and 150 microns, and/or at most about any of the following: 300, 250, 200, and 150 microns. Additionally or alternatively, the elastic film may comprise from 1 to about 8 individual layers, such as at least about any of the following: 1, 2, 3, and 4 layers, and/or at most about any of the following: 8, 7, 6, 5, and 4 layers. Additionally or alternatively, the elastic film may have a basis weight from about 10 to about 80 gsm, such as at least about any of the following: 10, 15, 20, 25, 30, 35, and 40 gsm, and/or at most about any of the following: 80, 75, 70, 65, 60, 55, 50, 45, and 40 gsm.

In accordance with certain embodiments of the invention, the elastic film comprises one or more elastomers such as an acrylate; a polyolefin, such as polyethylene, polypropylene, polybutylene, polyhexene, polyoctene; polystyrenes; polyurethanes; polyesters, such as polyethyleneterephthalate; polyamides such as nylon; natural or synthetic rubber resins such as styrenic block copolymers (e.g., styrene-isoprene-styrene, styrene-butadiene-styrene, styrene-ethylene-ethylene-propylene-styrene copolymers, styrene-ethylene-butylene-styrene); epoxies; vinyl acetates, such as ethylene vinyl acetate; polydiorganosiloxane polyurea copolymers; copolymers thereof and mixtures thereof.

The PEL, in accordance with certain embodiments of the invention, may have a total basis weight from about 10 to about 200 gsm, such as at least about any of the following: 10, 12, 20, 30, 40, 50, 60, 70, 80, 90, and 100 gsm, and/or at most about any of the following: 200, 190, 180, 170, 160, 150, 140, 130, 120, 110, and 100 gsm.

In accordance with certain embodiments of the invention, the PEL is devoid of an adhesive. Additionally or alternatively, the PEL may have a film coverage of at least about 80%, such as at least about any of the following: 80, 85, and 90% and/or at most about any of the following: 100, 99, 98, 97, 96, 95, 94, 93, 92, 91, and 90%. Additionally or alternatively, the PEL may have an elongation at break from about 250 to about 500%, such as at least about any of the following: 250, 280, 300, 320, and 350%, and/or at most about any of the following: 500, 480, 450, 420, 400, 380, and 350%. Additionally or alternatively, the PEL may have an elongation at break (%)-to-basis weight (gsm) ratio from about 4 to about 10, such as at least about any of the following: 4, 4.2, 4.5, 4.8, 5, 5.2, 5.5, 5.8, and 6, and/or at most about any of the following: 10, 9.5, 9, 8.5, 8, 7.5, 7, 6.5, and 6.

In accordance with certain embodiments of the invention, the PEL may comprise a plurality of apertures extending through the PEL. The plurality of apertures may define an open area for the PEL when in a relaxed state (e.g., non-stretched) from about 3 to about 30%, such as at least about any of the following: 3, 5, 8, 10, 12, 15, 18, and 20%, and/or at most about any of the following: 30, 28, 25, 22, and 20%.

In another aspect, the present invention provides a disposable absorbent article including at least one component thereof comprising a PEL, such as described and disclosed herein. The at least one component thereof may comprise an elastic ear, a panel, or a waistband, for example, of a diaper.

In accordance with certain embodiments of the invention, the disposable absorbent article comprises a disposable diaper, in which the disposable diaper comprises (i) a longitudinal direction and a transverse direction; (ii) a longitudinal centerline and a transverse centerline; (iii) an anterior region, a posterior region, and a central region positioned between the anterior region and the posterior region; (iv) a pair of elastic ear panels, the pair of elastic ear panels extending outwardly from transversely opposite side edges of the posterior region and comprising the PEL.

In accordance with certain embodiments of the invention, the disposable absorbent article comprises a disposable diaper, in which the disposable diaper comprises (i) a longitudinal direction and a transverse direction; (ii) a longitudinal centerline and a transverse centerline; and (iii) an anterior region, a posterior region, and a central region positioned between the anterior region and the posterior region; wherein the at least one of the anterior region and the posterior region have a waist portion comprising the PEL.

In another aspect, the present invention provides a method of making a PEL, such as those described and disclosed herein. The method may comprise the following steps: (a) providing or forming a first nonwoven layer; (b) melt extruding an elastic film onto the first nonwoven layer; (c) laminating the elastic film onto the first nonwoven layer by passing the clastic film and the first nonwoven layer through a roller nip comprising a point bond nip roll to form an intermediate material, wherein the point bond nip roll includes a first plurality of discrete protrusions that form a first plurality of discrete bond sites between the first nonwoven layer and a first side of the elastic film, and wherein the first plurality of discrete bond sites define a first bonded area and a first bond pattern between the first nonwoven layer and the first side of the elastic film, and wherein first nonbonded portions of the first nonwoven layer located between the first plurality of discrete bond sites are not bonded to the first side of the elastic film; and (d) activating the intermediate material to form the PEL, wherein activating the intermediate material forms deformed portions that project outwardly from an x-y plane.

In accordance with certain embodiments of the invention, the method may further comprise providing or forming a second nonwoven layer, and laminating the first nonwoven layer, the clastic film, and the second nonwoven layer together. For example, the elastic film may be positioned or located between and/or adjacent the first nonwoven layer and the second nonwoven layer. The method may comprise laminating the first nonwoven layer, the clastic film, and the second nonwoven layer together by passing the first nonwoven layer, the elastic film, and the second nonwoven layer through the roller nip. As noted above, the roller nip may comprise a point bond nip roller that also forms a second plurality of discrete bond sites between the second nonwoven layer and a second side of the elastic film. Alternatively, the roller nip may be defined by two separate point bond nip rolls through which the first nonwoven layer, the clastic film, and the second nonwoven layer are passed. Regardless, the second plurality of discrete bond sites define a second bonded area and a second bond pattern between the second nonwoven layer and the second side of the elastic film, and wherein second nonbonded portions of the second nonwoven layer located between the second plurality of discrete bond sites are not bonded to the second side of the elastic film.

The step of activating the intermediate material, in accordance with certain embodiments of the invention, may comprise forming deformed portions in both the first nonwoven layer and the second nonwoven layer that project outwardly from an x-y plane. In accordance with certain embodiments of the invention, activating the intermediate material may comprise incrementally stretching the intermediate material in a machine-direction, a cross-direction, or both. In accordance with certain embodiments of the invention, the activation may be carried out in a single step. Alternatively, the activation may be carried out over a series of multiple activation steps. For example, the intermediate material may be incrementally stretched in an initial activation step followed by subsequent activation steps. By way of example, the intermediate material may be subjected to from 2 to about 5 separate cross-stretching activation steps (e.g., 2, 3, 4, or 5) and/or from 2 to 5 separate machine direction-stretching activation step (e.g., 2, 3, 4, or 5).

FIG. 5 is a schematic illustrating the impact of an activation step to form a PEL 1 from an intermediate material 3, in accordance with certain embodiments of the invention. As illustrated by FIG. 5, the intermediate material 3 may include the first and second plurality of bond sites but may be devoid of the outwardly projecting deformed portions, while the outwardly projecting deformed portions 14, 34 may be imparted via the activation step.

In accordance with certain embodiments of the invention, the point bond nip roll comprises a rubber material or a metal material. The roller nip, for example only, may comprise the point bond nip roll and a backing roll. For instance, the backing roll may comprise a smooth surface roller.

In accordance with certain embodiments of the invention, the roller nip has a nip pressure from about 0 to about 15 psi, such as at least about any of the following: 0, 2, 3, 5, 6, and 8 psi, and/or at most about any of the following: 15, 14, 12, 10, and 8 psi. Additionally or alternatively, the compression provided by the roller nip may be controlled by setting a small gab between the nip roll and the backing roll (e.g., rubber roll). For example, the gap may be slightly less than the total thickness of the material (e.g., laminate or composite).

The method, in accordance with certain embodiments of the invention, may comprises preheating the first nonwoven layer, the second nonwoven layer, or both prior to the laminating step. For example, preheating the first nonwoven layer, the second nonwoven layer, or both may comprise heating the material in question to a temperature that is close or slightly higher than 25° C. This temperature, however, should generally not be more than 50° C. to avoid damaging the film thermally. Additionally or alternatively, the first nonwoven layer, the second nonwoven layer, or both are activated prior to the laminating step. Additionally or alternatively, the method may comprise a step of forming a plurality of apertures extending through the PEL. The plurality of apertures, if present, may define an open area of the PEL from about 2 to about 30%, such as at least about any of the following: 2, 3, 5, 6, 8, 10, 12, 14, and 15%, and/or at most about any of the following: 30, 28, 26, 25, 24, 22, 20, 18, 16, and 15%.

### EXAMPLES

The present disclosure is further illustrated by then following examples, which in no way should be construed as being limiting. That is, the specific features described in the following examples are merely illustrative and not limiting.

### Example Set 1

A PEL according to certain embodiments of the invention was compared to several commercially available materials used in ear applications or panels of diapers. The PEL was made from a 30 gsm elastic film sandwiched between two layers of nonwoven. Each layer of nonwoven was a 15 gsm spunbond (e.g., Kamisoft available from Berry Global, USA). This PEL was compared to commercial elastic side panels that were made of 50 gsm elastic film sandwiched between two layers of 30-35 gsm spunlace nonwovens or 22 gsm carded nonwovens. The commercial samples were made by adhesive lamination or ultrasonic lamination.

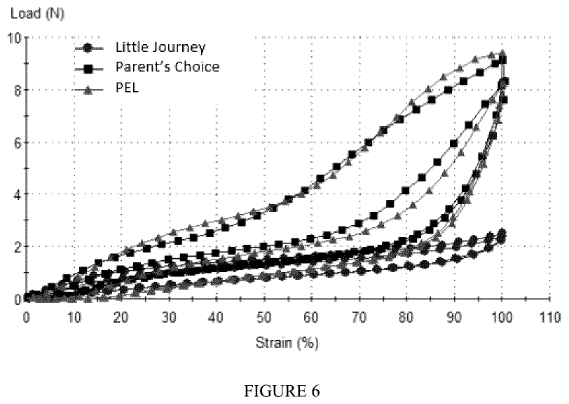

Table 1 below provides a summary of the measured physical properties of the PEL and the comparative commercially available materials. FIG. 6 illustrates a load vs. stain graph for the PEL and the comparative laminates that are commercially used as elastic ears or panels of disposable absorbent article (e.g., diaper).

TABLE 1

| | | | | | Panel |
|---|---|---|---|---|---|
| | | | | | Meijer Training |
| | | PEL | Back Ear Application | | Pants |
| | | Point Bond | Parents Choice | Little Journey | Adhesive |
| | | Extrusion | (Aplix) | (FQ/Berry) | (Apertured |
| Properties | | Lamination | Adhesive | Ultrasonic | Elastic Film) |
| Cost | Basis weight | 59 | 107 | 136 | 96 |
| | (gsm) | | | | |
| | % Film | 100 | 68-75 | 68-75 | 100 |
| | Coverage* | | | | |
| Strength | Elongation @ | 397 | 262 | 336 | 267 |
| | Break (%) | | | | |
| | Tensile @ | 11 | 22 | 37 | 17 |
| | Break (N) | | | | |
| | Tensile @ | 9 | 9 | 1 | 4 |
| | 100% (N) | | | | |
| Stretch | Extension @ | 50 | 42 | 90 | 70 |
| | 1000 g (%) | | | | |
| | Force Wall | 50 | 50 | 100 | NA |
| | (%) | | | | |
| Fit | Return Load | 0.85 | 1.4 | 0.78 | 0.6 |
| | @ 50% (N) | | | | |
| | Permanent | 11 | 4 | 4 | 8 |
| | Set (%) | | | | |
| Feel | Softness | ++ | +++ | + | +++ |
| Breathability | Airflow (cf) | 0 | 0 | 0 | 100 |
| Appearance | Visual | Rugosity | Varies | Varies | Varies |
| | Stretch | | | | |
| | Trigger | | | | |

### Example Set 2

A PEL according to certain embodiments of the invention was compared to several commercially available materials used in ear applications or panels of diapers. The PEL was made from a 30 gsm elastic film sandwiched between two layers of nonwoven. Each layer of nonwoven was a 15 gsm spunbond (e.g., Kamisoft available from Berry Global, USA). This PEL was compared to commercial elastic side panels that were made of 50 gsm elastic film sandwiched between two layers of 30-35 gsm spunlace nonwovens or 22 gsm carded nonwovens. The commercial samples were made by adhesive lamination or ultrasonic lamination.

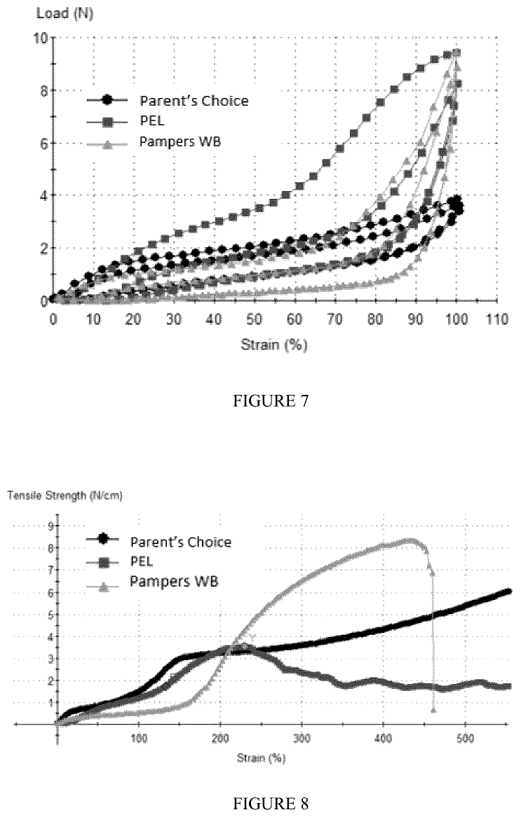

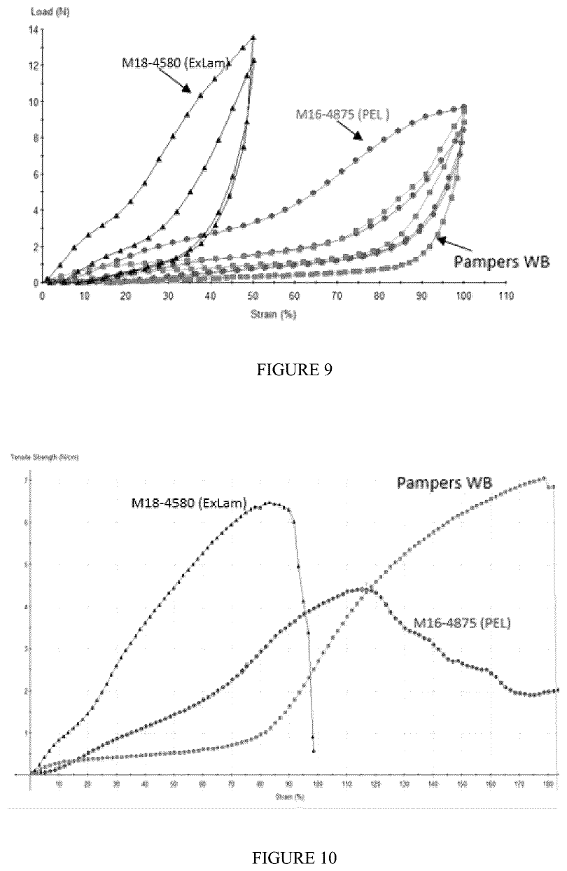

Table 2 below provides a summary of the measured physical properties of the PEL and the comparative commercially available materials. FIG. 7 illustrates a load vs. stain graph for a PEL and comparative laminates that are commercially used as waistbands of disposable absorbent article (e.g., diaper). FIG. 8 illustrates a tensile strength vs. strain graph for a PEL and comparative laminates that are commercially used as waistbands of disposable absorbent article (e.g., diaper). FIG. 9 illustrates a load vs. stain graph for a PEL and comparative laminates that are commercially used as waistbands of disposable absorbent article (e.g., diaper). FIG. 10 illustrates a tensile strength vs. strain graph for a PEL and comparative laminates that are commercially used as waistbands of disposable absorbent article (e.g., diaper).

TABLE 2

| | | PEL | | | |
|---|---|---|---|---|---|
| | | Point Bond | | Meijer | |
| | | Extrusion | Pampers WB | Training Pant | Parents Choice |
| Properties | | Lamination | Ultrasonic | Adhesive | Film Only |
| Cost | Basis weight | 59 | 80 | 69 | 46 |
| | (gsm) | | | | |
| | % Film | 100 | >90 | 100 | 100 |
| | Coverage* | | | | |
| Strength | Elongation @ | 397 | 205 | 131 | 750 |
| | Break (%) | | | | |
| | Tensile @ | 11 | 8 | 11 | 24 |
| | Break (N) | | | | |
| | Tensile @ | 9 | 7 | 9 | 4 |
| | 100% (N) | | | | |
| Stretch | Extension @ | 50 | 95 | NA | NA |
| | 1000 g (%) | | | | |
| | Force Wall (%) | 50 | | NA | NA |
| Fit | Return Load | 0.85 | 0.3 | 0.61 | 0.9 |
| | @ 50% (N) | | | | |
| | Permanent | 11 | 8.5 | 4 | 4 |
| | Set (%) | | | | |
| Feel | Softness | ++ | + | NA | NA |
| Breathability | Airflow (cf) | 0 | 0 | 0 | 0 |
| Appearance | Visual Stretch | Rugosity | Rugosity | flat | flat |
| | Trigger | | | | |

These and other modifications and variations to the invention may be practiced by those of ordinary skill in the art without departing from the spirit and scope of the invention, which is more particularly set forth in the appended claims. In addition, it should be understood that aspects of the various embodiments may be interchanged in whole or in part. Furthermore, those of ordinary skill in the art will appreciate that the foregoing description is by way of example only, and it is not intended to limit the invention as further described in such appended claims. Therefore, the spirit and scope of the appended claims should not be limited to the exemplary description of the versions contained herein.

What is claimed is:

1. A point bonded extrusion laminate (PEL), comprising:
(a) a first nonwoven layer;
(b) an elastic film;
(c) a first plurality of discrete bond sites bonding the first nonwoven layer to a first side of the elastic film, wherein the first plurality of discrete bond sites define a first bonded area and a first bond pattern between the first nonwoven layer and the first side of the elastic film; and
(d) first nonbonded portions of the first nonwoven layer located between the first plurality of discrete bond sites, wherein the first nonbonded portions of the first nonwoven layer are not bonded to the first side of the elastic film.

2. The PEL of claim 1, further comprising a second nonwoven layer, wherein the elastic film is located between the first nonwoven layer and a second nonwoven layer.

3. The PEL of claim 2, further comprising a second plurality of discrete bond sites bonding the second nonwoven layer to a second side of the elastic film, wherein the plurality of second discrete bond sites define a second bonded area and a second bond pattern between the second nonwoven layer and the second side of the elastic film, and second nonbonded portions of the second nonwoven layer located between the second plurality of discrete bond sites, wherein the second nonbonded portions of the second nonwoven layer are not bonded to the second side of the elastic film.

4. The PEL of claim 3, wherein the first nonwoven layer, the second nonwoven layer, or both comprise a spunbond nonwoven, a carded nonwoven, a meltblown nonwoven, a hydroentangled nonwoven, or any combination thereof.

5. The PEL of claim 4, wherein the first nonwoven layer, the second nonwoven layer, or both comprise a basis weight from about 3 to about 50 gsm.

6. The PEL of claim 3, wherein at least a portion of the first nonbonded portions, the second nonbonded portions, or both comprise deformed portions that project outwardly from an x-y plane.

7. The PEL of claim 6, wherein the deformed portions have an average height of about 1 to about 10 mm.

8. The PEL of claim 3, wherein the first bonded area, the second bonded area, or both comprise from about 5 to about 40%.

9. The PEL of claim 1, wherein the elastic film comprises an average thickness from about 10 to about 300 microns.

10. The PEL of claim 9, wherein the elastic film comprises one or more elastomers such as an acrylate; a polyolefin, such as polyethylene, polypropylene, polybutylene, polyhexene, polyoctene; polystyrenes; polyurethanes; polyesters, such as polyethyleneterephthalate; polyamides such as nylon; natural or synthetic rubber resins such as styrenic block copolymers (e.g., styrene-isoprene-styrene, styrene-butadiene-styrene, styrene-ethylene-ethylene-propylene-styrene copolymers, styrene-ethylene-butylene-styrene); epoxies; vinyl acetates, such as ethylene vinyl acetate; polydiorganosiloxane polyurea copolymers; copolymers thereof and mixtures thereof.

11. The PEL of claim 9, wherein the elastic film comprises from 1 to about 8 individual layers.

12. The PEL of claim 1, wherein the PEL is devoid of an adhesive.

13. The PEL of claim 1, wherein PEL has a film coverage of at least about 80%.

14. The PEL of claim 1, wherein the PEL has an elongation at break from about 250 to about 500%.

15. A disposable absorbent article, comprising: a disposable diaper including at least one component thereof comprising a PEL according to claim 1.

16. The article of claim 15, wherein the at least one component thereof comprises an elastic ear, a panel, or a waistband.

17. The article of claim 16, wherein the disposable diaper comprises (i) a longitudinal direction and a transverse direction; (ii) a longitudinal centerline and a transverse centerline; (iii) an anterior region, a posterior region, and a central region positioned between the anterior region and the posterior region; (iv) a pair of elastic ear panels, the pair of elastic ear panels extending outwardly from transversely opposite side edges of the posterior region and comprising the PEL.

18. The article of claim 16, wherein the disposable diaper comprises (i) a longitudinal direction and a transverse direction; (ii) a longitudinal centerline and a transverse centerline; and (iii) an anterior region, a posterior region, and a central region positioned between the anterior region and the posterior region; wherein the at least one of the anterior region and the posterior region have a waist portion comprising the PEL.

19. A method of making a point bonded extrusion laminate (PEL), comprising:
(a) providing or forming a first nonwoven layer;
(b) melt extruding an elastic film onto the first nonwoven layer;
(c) laminating the elastic film onto the first nonwoven layer by passing the elastic film and the first nonwoven layer through a roller nip comprising a point bond nip roll to form an intermediate material, wherein the point bond nip roll includes a first plurality of discrete protrusions that form a first plurality of discrete bond sites between the first nonwoven layer and a first side of the elastic film, and wherein the first plurality of discrete bond sites define a first bonded area and a first bond pattern between the first nonwoven layer and the first side of the elastic film, and wherein first nonbonded portions of the first nonwoven layer located between the first plurality of discrete bond sites are not bonded to the first side of the elastic film; and
(d) activating the intermediate material to form the PEL, wherein activating the intermediate material forms deformed portions that project outwardly from an x-y plane.

20. The method of claim 19, further comprising providing or forming a second nonwoven layer, and laminating the first nonwoven layer, the clastic film, and the second nonwoven layer together, wherein the clastic film is located between the first nonwoven layer and the second nonwoven layer.
